(19) United States
(12) Patent Application Publication
(10) Pub. No.: US 2016/0175329 A1
(43) Pub. Date: Jun. 23, 2016

(54) MESALAMINE SUPPOSITORY

(71) Applicant: Gavis Pharmaceuticals, Somerset, NJ (US)

(72) Inventor: Bala Chandran Nayar, Somerset, NJ (US)

(73) Assignee: GAVIS PHARMACEUTICALS, Somerset, NJ (US)

(21) Appl. No.: 14/578,033

(22) Filed: Dec. 19, 2014

(51) Int. Cl. A61K 31/606; A61K 47/44; A61K 9/02; A61J 3/08; A61K 45/06; A61K 9/00

(52) U.S. Cl. CPC A61K 31/606; A61K 45/06; A61K 9/0031; A61K 9/02; A61J 3/08; A61K 47/44

(57) ABSTRACT

A mesalamine rectal suppository having improved comfort of use and better retention in lower rectum for a long period of time is provided. The suppository contains a suppository base, at least one surfactant and at least one mucoadhesive agent. A method for manufacturing the suppository and methods for treating ulcerative colitis, such as active ulcerative proctitis, using such suppository is also provided.

### BACKGROUND OF THE INVENTION

(a) Field of the Invention

The present invention is directed to a mesalamine rectal suppository designed to provide improved comfort of use and better retention in the lower rectum for a long period of time. The invention is further directed to a method for manufacturing the suppository, and methods for treating ulcerative colitis, such as active ulcerative proctitis.

(b) Description of the Related Art

Inflammatory bowel diseases (IBD), such as Crohn's disease and ulcerative colitis (UC), are characterized by chronic, relapsing intestinal inflammation. Crohn's disease and UC are believed to involve a dysregulated immune response to gastrointestinal (GI) tract antigens, a mucosal barrier breach, and/or an adverse inflammatory reaction to a persistent intestinal infection. In healthy individuals without IBD, the GI tract luminal contents and bacteria constantly stimulate the mucosal immune system, and a delicate balance of pro-inflammatory and anti-inflammatory cells and molecules maintains the integrity of the GI tract, without eliciting severe and damaging inflammation [MacDermott, R. P., J Gastroenterology, 31:907:-916 (1996)]. It is unknown how the IBD inflammatory cascade begins, but constant GI antigen-dependent stimulation of the mucosal and systemic immune systems perpetuates the inflammatory cascade and drives lesion formation.

UC is a non-specific inflammatory disease of the colon that is of unknown cause and is characterized by diarrhoea with discharge of mucus and blood, cramping abdominal pain, inflammation and edema of the mucous membrane with patches of ulceration. UC limited to the rectum is known as ulcerative proctitis. People suffering from chronic UC affecting the whole colon have an increased risk of colonic cancer. Furthermore, when medical therapy fails, surgical resection of the affected bowel may be necessary.

In patients with more extensive disease, blood loss from the inflamed intestines can lead to anaemia, and may require treatment with iron supplements or even blood transfusions. Although infrequent, the colon can acutely dilate to a large size when the inflammation becomes very severe. This condition is called toxic megacolon. Patients with toxic megacolon are extremely ill with fever, abdominal pain and distention, dehydration, and malnutrition. Unless the patient improves rapidly with medication, surgery is usually necessary to prevent colon rupture and high risk of death.

Mesalamine, 5-aminosalicylic acid (5-ASA), is often used to treat UC and is effective in reducing disease symptoms and the incidence of relapse in UC. While mesalamine is available in oral form, intrarectal administration of it has several advantages. For example, rectal administration of a drug avoids some side-effects, such as gastrointestinal disorders, due to oral administration. As mesalamine is a locally GI active drug, lower doses of the drug can be administered rectally to obtain a better or equivalent therapeutic effect as that attained with a higher dose oral formulation. The absorption of a drug orally administered may also be affected by whether it is administered before or after each meal or between meals. There is no such food effect when drugs are administered intrarectally. Intrarectal administration can be performed even during periods of nausea, vomiting or unconsciousness, or after surgical procedures.

A mesalamine suppository is currently marketed in the United States by Aptalis Pharma as Canasa® for the treatment of active ulcerative proctitis.

U.S. Pat. No. 5,629,012 discloses a process of preparing a mesalamine suppository by preparing granulates of mesalamine, talc, magnesium stearate, polyvinyl pyrrolidone and polyethylene glycol and compressing the granulates in a tableting machine. The suppository contains 50-75% drug load. The patent further teaches to use a polyethylene glycol base as it is critical to form a uniform and smooth suppository with such a high drug load.

U.S. Pat. Nos. 8,217,083 and 8,436,051 disclose a suppository with an oily or fatty base, mesalamine of specific tap density, and a total drug load in the range of 35% to 50%.

It is considered advantageous to have a suppository of a size minimized as much as possible in order to ensure good patient compliance. The prior art teaches using mesalamine of a specific tap density and less drug load in order to achieve a small size suppository. The reduced drug load was achieved by using a substantial amount of hard fat. Advantages of such a formulation however can be extended only up to improved compliance by reducing the size of suppository.

Mesalamine undergoes rapid and extensive hepatic first-pass metabolism following oral administration. Currently approved suppositories of mesalamine contain a very high dose considering the demand for effective ulcerative colitis treatment. Rectal administration of mesalamine in such a high dose may result in a significant amount of the drug undergoing first pass metabolism.

There is a need for mesalamine suppositories which provide increased comfort of use as well as better retention in the lower rectum for a long period of time and as a result exhibit better bioavailability of the mesalamine.

### SUMMARY OF THE INVENTION

The present invention provides rectal suppositories of mesalamine. The suppository comprises a combination of a suppository base, a surfactant and a mucoadhesive agent. The suppository has a relatively smaller size and provides better retention in the rectum for a long period of time. Further, the suppository also delivers a therapeutically effective amount of mesalamine for the ulcerative colitis therapy by reducing first pass metabolism of the mesalamine. A method for treating ulcerative colitis with a such mesalamine suppository is further provided by the invention.

In one aspect, the invention provides a mesalamine rectal suppository comprising mesalamine, a suppository base, at least one surfactant, and at least one mucoadhesive agent.

In another aspect, the invention provides a mesalamine rectal suppository comprising mesalamine, a suppository base, at least one surfactant, and one or more mucoadhesive agents, wherein the suppository has a drug load ranging from 30% to 65%.

In another aspect, the invention provides a mesalamine rectal suppository comprising mesalamine, a suppository base, at least one surfactant, and one or more mucoadhesive agents, wherein the suppository comprises from about 450 to about 2,000 mg of mesalamine.

In another aspect, the invention provides a mesalamine rectal suppository comprising mesalamine, a suppository base, at least one surfactant, and one or more mucoadhesive agents, wherein the suppository comprises from about 0.5% w/w to about 5% w/w of a mucoadhesive agent. In an embodiment, the suppository comprises from about 0.5% w/w to about 2% w/w of the mucoadhesive agent.

In another aspect, the invention provides a mesalamine rectal suppository comprising mesalamine, a suppository base, at least one surfactant, and one or more mucoadhesive agents, wherein the mesalamine has a mean particle size of less than 120 microns. In an embodiment, 50% of the total amount of the mesalamine has a mean particle size of less than 20 microns. In a further embodiment, 50% of the total amount of the mesalamine has a mean particle size of less than 100 microns.

In another aspect, the invention provides a method of manufacturing a mesalamine rectal suppository, which process comprises the steps of:
(a) melting of hard fat (hydrogenated vegetable oil) in a suitable vessel fitted with a high shear mixer and a heating/cooling jacket;
(b) addition of a surfactant to step (a);
(c) addition of a mucoadhesive agent to step (b);
(d) addition of mesalamine to step (c);
(e) cooling the mixture of step (d);
(f) transferring the mixture of step (e) into a suppository moulding machine hopper; and
(g) filling the mixture of step (f) into the suppository mould, cooling and cutting into suitable strips.

Each step of addition involves mixing under low or high shear for a suitable period of time. The mixing during each step of addition can be performed for a period of 15 minutes to 2 hours. The sequence of addition steps can be interchangeable.

In another aspect, the invention provides a method of treating active ulcerative proctitis in a patient in need thereof comprising administering the mesalamine rectal suppository as substantially described herein. In an embodiment, the mesalamine rectal suppository is administered once a day for the treatment of active ulcerative proctitis.

Still other aspects and advantages of the invention will be apparent from the following detailed description of the invention.

### DETAILED DESCRIPTION OF THE INVENTION

The invention provides for a rectal suppository of mesalamine or salts thereof comprising a suppository base, at least one surfactant, and at least one mucoadhesive agent.

In another aspect, the invention provides for a rectal suppository that includes mesalamine as the active ingredient and pharmaceutical excipients that consist essentially of a suppository base, at least one surfactant, and at least one mucoadhesive agent to deliver the suppository with improved bioavailability and have a melting point that is less than about 37° C.

In another aspect, the invention provides for a rectal suppository consisting of a combination of an aminosalicylate and one or more of a local anaesthetic agent, a corticosteroid, and an immunosuppressant as the active ingredients and pharmaceutical excipients that consist of a suppository base, at least one surfactant, and at least one mucoadhesive agent to deliver the suppository with improved bioavailability and have a melting point that is less than about 37° C.

In another aspect, the invention provides for a rectal suppository consisting essentially of or consisting of a combination of an aminosalicylate and one or more of a local anaesthetic agent, a corticosteroid, and an immunosuppressant as the active ingredients and pharmaceutical excipients that consist essentially of a suppository base, at least one surfactant, and at least one mucoadhesive agent to deliver the suppository with improved bioavailability and have a melting point that is less than about 37° C.

The inventors have found that the combination of surfactant, mucoadhesive agent and the base in the suppository improved rectal bioavailability of mesalamine as the mucoadhesive force increased. Retaining mesalamine at the dosed site in the rectum by the addition of the mucoadhesive appeared to be very important in voiding first-pass hepatic elimination and increasing the bioavailability of mesalamine.

The inventors have further observed that use of two types of mesalamine particles differing in particle sizes is also advantageous to improve bioavailability and additionally the size of the resulting suppository can be kept smaller.

The term “mesalamine,” as used herein, includes its base, pharmaceutically acceptable salts, optical isomers and racemic mixtures.

The term “drug load” refers to the weight percentage of mesalamine based on the total weight of the suppository.

The invention provides a mesalamine rectal suppository comprising mesalamine, a suppository base, and at least one mucoadhesive agent.

The amount of mesalamine in the suppository ranges from about 450 to about 2,000 mg of mesalamine. In an embodiment, the suppository has a drug load ranging from about 30% to about 65%. In a further embodiment, the suppository has a drug load ranging from about 50% to about 65%.

In an embodiment, mesalamine particles have a mean particle size of less than 120 microns. Preferably, 30% to 60% of the total amount of the mesalamine has a mean particle size of less than 20 microns and 40% to 70% of the mesalamine has a mean particle size of less than 100 microns.

Suitable mucoadhesive agents, by way of example and without limitation, include polycarbophil, carbopol, sodium alginate, hydroxyethyl cellulose, hydroxypropyl cellulose, carboxymethyl cellulose, natural gums, lecithins, pectin, ammonia alginate, calcium alginate, potassium alginate, propylene glycol alginate, agar, gum arabic, gum karaya, locust bean gum, tragacanth, carrageenans, guar, xanthan, and scleroglucan, and mixtures thereof. Preferred mucoadhesive agents are selected from polycarbophil, carbopol, sodium alginate, and hydroxyethyl cellulose.

In an embodiment, the amount of mucoadhesive agent in the suppository is from about 0.5% w/w to about 5% w/w. Preferably the amount of mucoadhesive agent in the suppository is from about 0.5% w/w to about 2% w/w.

The suppository base can be an oily or fatty base. Conventional suppository bases which may be employed include theobroma oil, cocoa butter, hard fats, glycerides of fatty acids, glycerol-gelatin bases, and mixtures thereof. Suitable hard fat bases include, but are not limited to, esterified mixtures of mono-, di- and triglycerides which are obtained by esterification of fatty acids (European Pharmacopoeia, 3rd edition 1997, Deutscher Apotheker Verlag Stuttgart. p. 1022; The United States Pharmacopoeia, USP 23, NF18). Such hard fats are commercially available, for example, under the name Witepsol™ (e.g. Witepsol™ H12 and H15). A preferred suppository base is hard fat (e.g., hard fat NF).

Preferred hard fat bases include, but are not limited to, hard fats containing a mixture of mono-, di- and triglycerides of saturated C₉₋₁₈ fatty acids or the mixture of triglycerides, diglycerides and monoglycerides. It is a synthetic fat prepared by hydrogenating suitable vegetable oils. The hard fat base can comprise hard fats obtained by esterification of fatty acids of vegetable origin with glycerol, a macrogol ether containing 20 to 24 oxyethylene groups in the polyoxyethylene chain, e.g., polyoxyl-20-cetostearyl ether, and glycerides, e.g., glyceryl ricinoleate. A preferred hard fat is hydrogenated vegetable oil.

Other suitable suppository bases include, but are not limited to, cocoa butter, lauric oil, beef tallow, hard fat, and any combination of any of the foregoing.

In an embodiment, the suppository base is devoid of polyethylene glycol.

The suppository may further contain a surfactant to improve mesalamine dispersing into the oily or fatty base, to increase the spreading of the melted suppository on the rectal mucosa leading to a greater contact surface, and to reduce the viscosity of the molten mass and to reduce the pathway of drug particles to the interface.

Suitable surfactants, by way of example and without limitation, include disodium laurenth sulfosuccinate, polysorbates, polyoxyethylene derivatives of natural or hydrogenated vegetable oils such as castor oil; polyoxyethylene-sorbitan fatty acid esters, such as mono-, di- and tri-lauryl, palmityl, stearyl and oleyl esters; alkyl/dialykyl sulfate, sulfonate or sulfosuccinate salts such as sodium lauryl sulfate and dioctyl sodium sulfosuccinate; polyoxyethylene fatty acid esters; phospholipids such as lecithins; transesterification products of natural vegetable oil triglycerides and polyalkylene polyols; sorbitan fatty acid esters; pentaerythritol fatty acid esters; polyoxyethylene glycol alkyl ethers and esters; and the like.

Examples of specific surfactants which may be used include, without limitation, polyoxyethylene castor oil derivatives, such as polyoxyethylene glycerol triricinoleate polyoxyl 35 castor oil (CREMOPHOR® EL, available from BASF Corporation), and polyoxyl 40 hydrogenated castor oil (CREMOPHOR® RH40, available from BASF Corporation); mono-fatty acid esters of polyoxyethylene (20) sorbitan, such as polyoxyethylene (20) sorbitan monooleate (TWEEN® 80), polyoxyethylene (20) sorbitan monostearate (TWEEN® 60), polyoxyethylene (20) sorbitan monopalmitate (TWEEN® 40), and polyoxyethylene (20) sorbitan monolaurate (TWEEN® 20) (all available from ICI Surfactants, Wilmington, Del.); polyoxyethylene glycol 200 monostearate (MYRJ® 52, available from Calgene Chemicals, Skokie, Ill.); polyglycerol esters with a HLB of 10 or greater, such as decablyceryl mono- and dioleate and the like; and mixtures thereof.

The surfactant may comprise about 0.2% to about 2% w/w of the suppository.

The invention further provides a method of manufacturing the mesalamine rectal suppository of the invention comprising matrix core. The process comprises the steps of:
(a) melting of hard fat (hydrogenated vegetable oil) in a suitable vessel fitted with a high shear mixer and a heating/cooling jacket;
(b) addition of a surfactant to step (a);
(c) addition of a mucoadhesive agent to step (b);
(d) addition of mesalamine to step (c);
(e) cooling the mixture of step (d);
(f) transferring the mixture of step (e) into a suppository moulding machine hopper; and
(g) filling the mixture of step (f) into the suppository mould, cooling and cutting into suitable strips.

Each step of addition involves mixing under low or high shear for suitable period of time. The mixing during each step of addition can be performed for a period of 15 minutes to 2 hours. The sequence of addition steps can be interchangeable.

The total weight of the suppository preferably ranges from about 2,000 to about 3,000 mg. According to one embodiment, the suppository has a total weight of about 2,000 mg, about 2,500 mg or about 2,900 mg.

The suppository is preferably smooth and torpedo-shaped.

The melting point of the suppository is generally sufficient to melt in the patient's body, and is typically no more than about 37° C.

The mesalamine suppository of the invention further may contain one or more additional therapeutic agents used for treating ulcerative colitis (such as active ulcerative proctitis). The additional therapeutic agents can be selected from aminosalicylates such as sulfazine, olsalazine, balsalazide; corticosteroids such as prednisone, methylprednisolone, budesonide; local anaesthetics such as benzocaine, lidocaine, bupivacaine; and immunosupressants such as azathioprine, 6-mercaptopurine, cyclosporine A, tacrolimus, methotrexate.

The mesalamine suppository can be administered to treat ulcerative colitis, such as active ulcerative proctitis, in a patient in need thereof. Preferably, the mesalamine suppository is administered in sufficient quantity and frequency to reduce the symptoms of ulcerative colitis.

The mesalamine suppository can also be administered prophylactically to a patient at risk for ulcerative colitis (such as active ulcerative proctitis). Preferably, the mesalamine suppository is administered in sufficient quantity and frequency to delay or prevent the onset of symptoms of ulcerative colitis (e.g., to delay or prevent the onset of abdominal pain, diarrhea, rectal bleeding, weight loss, fever, loss of appetite, dehydration, anemia, or malnutrition, or any combination thereof).

In the above methods, the mesalamine suppository is preferably administered once a day and more preferably once a day at bedtime. The suppository is also preferably retained for one to three hours or longer, if possible. The treatment can be brief, for example, once daily for three to twenty-one days, or can be longer, for example, once daily for three to six weeks.

### EXAMPLE 1

TABLE 1
Mesalamine Suppository with Mucoadhesive agent

| Sr. | | Formulation (Qty in mg) | | | |
|---|---|---|---|---|---|
| No. | Ingredients | 1 | 2 | 3 | 4 |
| 1 | Mesalamine | 400 | 600 | 800 | 0 |
| | (Micronized) | | | | |
| 2 | Mesalamine | 400 | 600 | 800 | 1000 |
| | (Non-Micronized) | | | | |
| 3 | Hard fat | 1170 | 750 | 830 | 1810 |
| | (Hydrogenated Vegetable Oil) | | | | |
| 4 | Surfactant | 10 | 20 | 30 | 40 |
| 5 | Mucoadhesive | 20 | 30 | 40 | 50 |
| | Agent | | | | |

| | | | | |
|---|---|---|---|---|
| TOTAL | 2000 | 2000 | 2500 | 2900 |

Procedure:
(a) melt the hard fat in a stainless steel tank fitted with a high shear mixer and a heating/cooling jacket at about 60° C.;
(b) add the surfactant to step (a) and mix under low shear for about 30 minutes;
(c) add the mucoadhesive agent to step (b) under high shear for about 30 minutes;
(d) add mesalamine to step (c) high shear for about 60 minutes;
(e) cool the mixture of step (d) to about 50° C.;
(f) transfer the mixture of step (e) into a suppository molding machine hopper (such as manufactured by Sarong, Italy) under constant mixing with a suitable mixer in the hopper;
(g) fill the molten mixture of step (f) into the suppository mould (either aluminium or plastic) and cool down to about 15° C. through various cooling chambers of the suppository manufacturing machine; and
(h) cut the suppositories of step (g) into suitable strips of 4, 6, 10, etc. as desired and collect.

What is claimed is:

1. A mesalamine rectal suppository comprising mesalamine, a suppository base, at least one surfactant and at least one mucoadhesive agent.

2. The mesalamine rectal suppository of claim 1, wherein the suppository comprises:
from about 0.5% w/w to about 5% w/w of the mucoadhesive agent; and
from about 0.2% w/w to up to 2% w/w of surfactant.

3. The mesalamine rectal suppository of claim 2, wherein the suppository comprises from about 0.5% w/w to up to 2% w/w of the mucoadhesive agent.

4. The mesalamine rectal suppository of claim 1, wherein the mesalamine has a mean particle size of less than 120 microns.

5. The mesalamine rectal suppository of claim 1, wherein 30% to 60% of the total amount of mesalamine in the suppository has a mean particle size of less than 100 microns.

6. The mesalamine rectal suppository of claim 1, wherein 40% to 70% of the total amount of mesalamine in the suppository has a mean particle size of less than 20 micron.

7. The mesalamine rectal suppository of claim 1, wherein the suppository has a drug load ranging from about 30% to about 65%.

8. The mesalamine rectal suppository of claim 1, wherein the suppository comprises from about 450 to about 2,000 mg of mesalamine.

9. The mesalamine rectal suppository of claim 1, wherein the surfactant is selected from the group consisting of disodium laurenth sulfosuccinate; polysorbates;
polyoxyethylene derivatives of natural or hydrogenated vegetable oils; polyoxyethylene-sorbitan fatty acid esters; alkyl/dialykyl sulfate, sulfonate or sulfosuccinate salts;
polyoxyethylene fatty acid esters; phospholipids; transesterification products of natural vegetable oil triglycerides and polyalkylene polyols; sorbitan fatty acid esters;
pentaerythritol fatty acid esters; and polyoxyethylene glycol alkyl ethers and esters; and
mixtures thereof.

10. The mesalamine rectal suppository of claim 1, wherein the mucoadhesive agent is selected from the group consisting of polycarbophil, carbopol, sodium alginate, hydroxyethyl cellulose, hydroxypropyl cellulose, carboxymethyl cellulose, natural gums, lecithins, pectin, ammonia alginate, calcium alginate, potassium alginate, propylene glycol alginate, agar, gum arabic, gum karaya, locust bean gum, tragacanth, carrageenans, guar, xanthan, and scleroglucan, and mixtures thereof.

11. The mesalamine rectal suppository of claim 1, wherein the suppository base is selected from the group consisting of theobroma oil, cocoa butter, oily or fatty base, glycerides of fatty acids, and glycerol-gelatin bases.

12. The mesalamine rectal suppository of claim 12, wherein the suppository base is oily or fatty base.

13. The mesalamine rectal suppository of claim 1, wherein the suppository base is devoid of polyethylene glycol.

14. The mesalamine rectal suppository of claim 1, wherein the suppository is a moulded suppository.

15. The rectal suppository of claim 1, wherein the suppository includes mesalamine as the sole active ingredient and consists essentially of a suppository base, at least one surfactant and at least one mucoadhesive agent to deliver the active ingredients.

16. The rectal suppository of claim 1, wherein the suppository consists of mesalamine and one or more of a local anaesthetic agent, a corticosteroid, and an immunosuppressant as the active ingredients and consists essentially of a suppository base, at least one surfactant and at least one mucoadhesive agent to deliver the active ingredients.

17. The rectal suppository of claim 1, wherein the suppository consists of mesalamine and one or more of a local anaesthetic agent, a corticosteroid, and an immunosuppressant as the active ingredients and consists of a suppository base, at least one surfactant and at least one mucoadhesive agent to deliver the active ingredients.

18. A rectal suppository consisting essentially of an aminosalicylate and one or more of a local anaesthetic agent, a corticosteroid, and an immunosuppressant as the active ingredients and consisting essentially of a suppository base, at least one surfactant and at least one mucoadhesive agent to deliver the active ingredients and have a melting point that is less than about 37° C., wherein:
the aminosalicylate is present in a first portion and a second portion, the first portion being micronized and having a first particle size, the second portion being non-micronized and having a second particle size, and the first particle size and the second particle size are different;
the suppository base comprises a hard fat;
the suppository has a total weight between about 2,000 and about 3,000 mg and a drug load from about 30% to about 65% of the total weight of the suppository; and
the suppository contains from about 0.5% w/w to about 5% w/w of the mucoadhesive agent, from about 0.2% w/w to about 2% w/w of the surfactant, from about 450 to about 2,000 mg of the aminosalicylate, and about 5 to about 100 mg of the local anaesthetic agent, the corticosteroid, or the immunosuppressant.

19. A method of treating active ulcerative proctitis in a patient in need thereof comprising administering the mesalamine rectal suppository of claim 1 to the patient.

20. A method of manufacturing the mesalamine rectal suppository of claim 1, which method comprises the steps of:
(a) melting of hard fat in a suitable vessel fitted with a high shear mixer and a heating/cooling jacket;
(b) addition of the surfactant to step (a);
(c) addition of the mucoadhesive agent to step (b);
(d) addition of the mesalamine to step (c);
(e) cooling the mixture of step (d);
(f) transferring the mixture of step (e) into a suppository moulding machine hopper; and
(g) filling the mixture of step (f) into the suppository mould, cooling and cutting into suitable strips.
